(19) United States
(12) Patent Application Publication
(10) Pub. No.: US 2004/0067926 A1
(43) Pub. Date: Apr. 8, 2004

(54) Sustained-release suspension of ceftiofur hydrochloride

(76) Inventors: Yong-Sik Kim, Yuseong-gu (KR); Je Phil Ryoo, Yuseong-gu Taejeon (KR); Mi-Suk Choi, Yuseong-gu Taejeon (KR)

Correspondence Address: Birch Stewart, Kolasch & Birch, PO Box 747, Falls Church, VA, 22040-0747, US

(21) Appl. No.: 10/467,095

(22) PCT Filed: Feb. 4, 2002
(86) PCT No.: PCT/KR02/00131

(30) Foreign Application Priority Data
Feb. 19, 2001 (KR) 2001/8164

(51) Int. Cl. A61K 31/545; A61K 31/355; A61K 35/78; A61K 9/22

(52) U.S. Cl. 514/200; 424/468; 424/757; 514/458

(57) ABSTRACT

Disclosed is a sustained-release suspension composition containing ceftiofur hydrochloride, tocopherol or a derivative thereof, and a biocompatible oil.

### TECHNICAL FIELD

The present invention relates to a sustained-release suspension containing ceftiofur or a pharmaceutically acceptable salt thereof as the active ingredient. More specifically, the invention relates to the sustained-release suspension containing ceftiofur or a pharmaceutically acceptable salt thereof, particularly, ceftiofur hydrochloride, tocopherol or a derivative thereof, and a biocompatible oil.

### BACKGROUND ART

Ceftiofur hydrochloride of the following formula (I) is a third generation cephalosporin antibiotic with a broad spectrum, having activity against Gram-positive and Gram-negative bacteria:

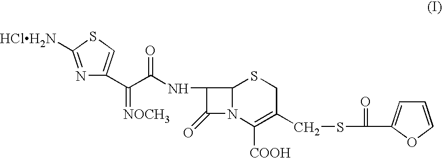

It displays an excellent therapeutic effect on diarrhea, pneumonia, transmissible gastroenteritis or mastitis of livestock, such as cattle and swine, or poultry, caused by Actinobacillus spp., Salmonella spp., Pasteurella spp., Streptococcus spp., Mycoplasma spp. or Haemophilus spp., etc. Because of its poor solubility in water, it should be formulated into a suspension, and then, subcutaneously or muscularly injected to animals. Generally, a suspension, as a pharmaceutical dosage form, should satisfy therapeutic effectiveness, physical and chemical stability, durability and appearance. Currently marketed suspensions of ceftiofur hydrochloride have short duration of the pharmacological effect, and thus should be administered daily for 3 days. In addition, in such suspensions, particles are settled down at a high rate and separation of layers occurs rapidly. Caking of the sediment layer makes resuspension difficult.

In order to improve the above-described problem in resuspension, it is known to be effective to add electrolytes [Patel, The Theory and Practice of Industrial Pharmacy], surfactant [Nash, Pharmaceutical Dosage Forms: Disperse Systems, vol. 1], water (PCT/WO98/25621, U.S. Pat. No. 5,736,151), etc. Those substances were reported to prevent caking caused by agglomeration of particles thereby improving resuspendability.

In an oil-containing suspension of somatotropin, the addition of tocopherol acetate as an anti-oxidant, for preventing oxidation reaction of somatotropin, is known to be capable of increasing its durability (U.S. Pat. No. 5,520,927 and Korean Patent No. 177,306). However, such a suspension has an injectability problem, because its viscosity is remarkably increased as the temperature is decreased.

### DISCLOSURE OF THE INVENTION

The present inventors performed extensive studies to develop a sustained-release suspension of ceftiofur or a pharmaceutically acceptable salt thereof, particularly, ceftiofur hydrochloride, which provides convenience in administration with a prolonged pharmacological effect and is readily resuspendable. As a result, the inventors found that a suspension, containing ceftiofur or a pharmaceutically acceptable salt thereof, tocopherol or a derivative thereof, and a biocompatible oil, has a prolonged drug release time of 72 hours and a remarkably decreased sedimentation rate of particles, and thus is readily resuspendable. Therefore, the inventors completed the present invention. The suspension, containing ceftiofur or its pharmaceutically acceptable salt, tocopherol or a derivative thereof, and a biocompatible oil, is novel, since it has not yet been published in any literature.

An object of the present invention is to provide a sustained-release suspension of ceftiofur or a pharmaceutically acceptable salt thereof, which has a prolonged pharmacological effect and is readily resuspendable.

The present invention provides a sustained-release suspension composition containing ceftiofur or a pharmaceutically acceptable salt, tocopherol or a derivative thereof, and a biocompatible oil.

In the present invention, the pharmaceutically acceptable salt of ceftiofur includes, but is not specifically limited to, ceftiofur sodium, ceftiofur hydrochloride, etc. and the most preferable one is ceftiofur hydrochloride.

The biocompatible oil may be vegetable oil, animal oil or synthetic oil of any kind, which is not specifically limited, as long as it has neither any harmful effect nor irritation on the body. Preferable is vegetable oil, such as soybean oil, cottonseed oil, sesame oil, corn oil, olive oil, peanut oil, palm oil, or mixtures thereof, and the most preferable is soybean oil.

Tocopherol or a derivative thereof is generally used as an anti-oxidant. However, in the present invention, it is used as a suspending medium in combination with the biocompatible oil. Preferable is α-tocopherol, β-tocopherol, γ-tocopherol, δ-tocopherol, α-tocopherol succinate, or tocopherol acetate, for example, α-tocopherol acetate, and the most preferable is α-tocopherol acetate.

In the composition of the present invention, the content of the biocompatible oil is not specifically limited, but preferable is 50 to 90% by weight, and more preferable is 65 to 85% by weight. The content of tocopherol or the derivative thereof is not specifically limited, but preferable is 10 to 50% by weight and more preferable is 10 to 30% by weight.

The present composition contains a therapeutically effective amount of ceftiofur or a pharmaceutically acceptable salt thereof, particularly ceftiofur hydrochloride, such as preferably 0.1 to 20% and more preferably 1 to 10% by weight of ceftiofur hydrochloride.

The present composition may contain pharmaceutically acceptable excipients, for example, stabilizers or preservatives. The composition may be administered by intramuscular or subcutaneous injection to livestock, such as cattle or swine, or poultry.

The present composition may be produced by homogeneously mixing ceftiofur or a pharmaceutically acceptable salt thereof with a biocompatible oil, and tocopherol or a derivative thereof according to a conventional method for manufacturing a suspension.

The present composition, which contains ceftiofur or a pharmaceutically acceptable salt thereof, tocopherol or a derivative thereof, and a biocompatible oil, has a prolonged duration time of the pharmacological effect and a delayed sedimentation rate of particles, and thus resuspends easily. Such effects can be identified by Drug Dissolution Test in the following Experiment 1 and Resuspension Test in the following Experiment 2. In case of containing an excess of tocopherol or the derivative thereof, viscosity is remarkably increased, and sedimentation of particles and separation of layers are delayed, but sustaining effect of drug release is not considerable. However, the content of tocopherol or the derivative thereof has little relation with resuspendability.

### BRIEF DESCRIPTION OF THE DRAWINGS

FIG. 1 is a graph showing a cumulative dissolution rate of the drug in the present suspension.

FIG. 2 is a graph showing a resuspension rate of the drug in the present suspension.

### BEST MODE FOR CARRYING OUT THE INVENTION

This invention will be better understood from the following examples. However, one skilled in the art will readily appreciate the specific materials and results described are merely illustrative of, and are not intended to, nor should be intended to, limit the invention as described more fully in the claims, which follows thereafter. Ceftiofur hydrochloride used in the following examples has purity of 90% or more, and an average particle diameter of 1 to 5 μm after air-mill.

### EXAMPLE 1

The following ingredients were homogeneously mixed in a homo mixer to prepare a suspension:

| | |
|---|---|
| Ceftiofur hydrochloride | 5 g |
| Soybean oil | 80 g |
| Tocopherol acetate | 15 g |
| Total | 100 g |

### EXAMPLE2

A suspension was prepared using the following ingredients according to the substantially same method in Example 1:

| | |
|---|---|
| Ceftiofur hydrochloride | 5 g |
| Soybean oil | 75 g |
| Tocopherol acetate | 20 g |
| Total | 100 g |

The viscosity of suspensions prepared in the above Examples 1 and 2 was measured to be 30 to 150 cps at normal temperature. Therefore, the suspensions were evaluated to have no problem in injectability for injection.

### Experiment 1: Drug Dissolution Test

Drug dissolution rate of the suspensions obtained from Examples 1 and 2 was measured using a dissolution instrument. The currently marketed ceftiofur hydrochloride suspension (Excenel® manufactured by Pharmacia-Upjohn) was used as a control.

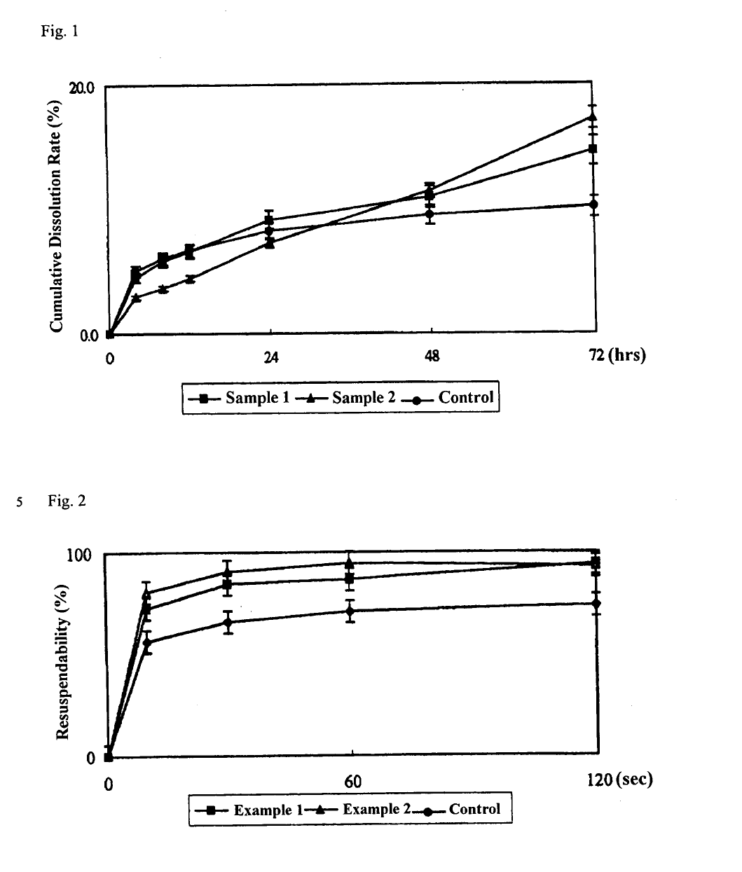

The dissolution test was carried out as follows. A mixed solution of polyethylene glycol and tertiary distilled water (50:50) of 5 ml was filled into a dissolution instrument connected with a thermostatic water bath at 37° C. In the above solution, a drug was dissolved from the suspensions obtained from the above Examples. Samples were taken at regular intervals and then, analyzed by HPLC. The dissolved amount of ceftiofur hydrochloride to the initial amount thereof in the samples was measured and expressed as the dissolution rate (%). The results are shown in FIG. 1.

As shown in FIG. 1, the control had a larger initial released amount of the drug than the suspensions of the Examples, but had the nearly unchanged cumulative dissolution amount with the lapse of time. In comparison, the drug was continuously released after 72 hours in the suspensions of the Examples. Therefore, it was concluded that the ceftiofur hydrochloride suspensions of the present invention had the controlled or sustained release of the drug and the prolonged release time of the drug to 72 hours.

### Experiment 2: Resuspension Test

In order to test resuspendability of the suspension, a resuspension test was carried out as follows. The currently marketed ceftiofur hydrochloride suspension (Excenel® manufactured by Pharmacia-Upjohn ) was used as a control.

The suspensions obtained from the above Examples 1 and 2, and the control were allowed to stand at normal temperature for 20 days. As a result, the suspensions of the Examples and the control were identified to have a similar particle diameter, i.e. 1 to 5 μm, but quite a different sedimentation rate. That is, distinct separation of layers was observed in the control after 24 hours, but no separation of layers was observed in the suspensions of the Examples even after 24 hours.

After 20 days, the suspensions were rotated and mixed using a rotary mixer. Samples were taken from the pre-determined part at regular intervals, and then, concentrations thereof were measured by HPLC. Resuspension rate was calculated from the measured concentration to the initial concentration. The results are shown in FIG. 2. As shown in FIG. 2, the control had the resuspension rate of 70 to 75% after rotating and mixing for 120 seconds. In comparison, the suspensions of Examples 1 and 2 had the resuspension rate of almost 100% after rotating and mixing for 120 seconds. The resuspension rate of the suspensions was not significantly varied depending on the content of tocopherol acetate, and generally higher than that of the control.

### EXAMPLES 3 and 4

Suspensions were prepared according to the substantially same method as Examples 1 and 2 except using cottonseed oil instead of soybean oil.

### Experiments 3 and 4

The same experiments in Experiments 1 and 2 were carried out using the suspensions obtained from Examples 3 and 4, and similar results were obtained.

### EXAMPLES 5 and 6

Suspensions were prepared according to the substantially same method as Examples 1 and 2 except using sesame oil instead of soybean oil.

### Experiments 5 and 6

The same experiments in Experiments 1 and 2 were carried out using the suspensions obtained from Examples 5 and 6. As a result, as compared with the suspensions of Examples 1 and 2, the above suspensions had a decreased sedimentation rate, but a reduced dissolution amount, and had some difficulty in resuspension. This may be because sesame oil has higher viscosity than soybean oil and cottonseed oil. Therefore, it was supposed that a sesame oil-containing suspension would have an excellent sustained release effect and resuspendability of the drug, if a content of the tocopherol derivative is reduced to an appropriate amount.

### INDUSTRIAL APPLICABILITY

The ceftiofur hydrochloride suspension of the present invention has prolonged duration of the drug of 72 hours and is readily resuspendable. Therefore, the present suspension displays a prolonged pharmacological effect only with a single administration. Therefore, it does not need to be successively administered like known formulations. In addition, the present suspension has a decreased sedimentation rate of particles and is readily resuspendable, and thus has high stability even after long-term storage, which is expected to reduce much cost.

What is claimed is:

1. A sustained-release suspension composition containing ceftiofur or a pharmaceutically acceptable salt thereof, tocopherol or a derivative thereof, and a biocompatible oil.

2. The composition according to claim 1, wherein the pharmaceutically acceptable salt of ceftiofur is ceftiofur hydrochloride.

3. The composition according to claim 1, wherein the biocompatible oil is selected from the group consisting of soybean oil, cottonseed oil, sesame oil, corn oil, olive oil, peanut oil and palm oil, and mixtures thereof.

4. The composition according to claim 3, wherein the biocompatible oil is soybean oil.

5. The composition according to claim 1, wherein tocopherol or the derivative thereof is selected from the group consisting of α-tocopherol, β-tocopherol, γ-tocopherol, δ-tocopherol, α-tocopherol succinate and tocopherol acetate, and mixtures thereof.

6. The composition according to claim 5, wherein the derivative of tocopherol is α-tocopherol acetate.

7. The composition according to claim 1, wherein the content of the biocompatible oil is in the range of 50 to 90% by weight.

8. The composition according to claim 1, wherein the content of tocopherol or the derivative thereof is in the range of 10 to 50% by weight.

9. The composition according to claim 2, wherein the content of ceftiofur hydrochloride is in the range of 0.1 to 20% by weight.
